(19) United States
(12) Patent Application Publication
(10) Pub. No.: US 2010/0042020 A1
(43) Pub. Date: Feb. 18, 2010

(54) Focused energy delivery apparatus method and system

(76) Inventor: Shmuel Ben-Ezra, Pardes-Hanna (IL)

Correspondence Address: FENNEMORE CRAIG, 3003 NORTH CENTRAL AVENUE, SUITE 2600, PHOENIX, AZ, 85012, US

(21) Appl. No.: 12/222,626

(22) Filed: Aug. 13, 2008

(51) Int. Cl. A61H 1/00; A61B 19/00

(52) U.S. Cl. 601/3; 128/898

(57) ABSTRACT

There is provided herein a system, method and an apparatus for delivering focused therapeutic energy, such as ultrasonic energy, the apparatus includes a transducer adapted to transmit focused energy to a target area tissue of a subject body and a positioning element adapted to shift the transducer while transmitting the focused energy to the target area tissue, wherein the positioning element is further adapted to substantially maintain a focal point of the transducer within the target area tissue of the subject body.

### FIELD

The invention relates to apparatus for performing focused energy-delivery treatment on tissues.

### BACKGROUND

Focused energy delivery is widely used in the medical field, both for diagnostic and therapeutic purposes. An example of an application of focused energy delivery may be seen in an ultrasound scanner, which is adapted to use ultrasound for diagnosing certain medical conditions such as tumors and renal stones, and for monitoring fetus development during pregnancy. Ultrasound generally comprises sound waves having a frequency greater than the typical upper limit of human hearing, which is around 20 kilohertz (kHz).

Therapeutic focused energy delivery (TFED) may be used for ablation and/or destroying of pathogenic objects and various tissues. TFED may also be used to destroy fat tissues, for example, in non-invasive body contouring procedures, where energy is selectively targeted to disrupt fat cells, essentially without damaging neighboring structures.

Generally, tissue destruction using TFED comprises subjecting the tissue to thermal and/or mechanical stresses. Thermal stresses are created by a temperature increase in a treatment area, obtained by direct absorption of ultrasonic energy in the tissues in the treatment area. The increased temperature causes damaging processes, such as coagulation, within the tissue. Mechanical stresses include streaming, shear forces, tension, and cavitation. These stresses cause fractionation, rupture and/or liquefaction of cells, which in turn may result in tissue destruction. Cavitation is a physical phenomenon in which bubbles are formed within the tissue. Cavitation near cells will damage or destroy many of the cells. The cavitation phenomenon depends on specific tissue characteristics when employed in a biological environment. This enables tissue differentiation for damage or destruction, which means that fat cells can be destroyed (or damaged sufficiently to die soon after), while blood vessels, peripheral nerves, skin, muscle and connective tissue within an energy focus, as well as neighboring tissues such as listed above outside the focus, will remain intact. Other destructive mechanisms, such as cell apoptosis, may also directly or indirectly be involved in the non-invasive focused energy treatment.

As previously discussed, TFED is generally used to deliver energy into a confined region inside a body for therapeutic purposes. For convenience hereinafter, the confined region may also be referred to as treatment area or target area. A TFED transducer, adapted to deliver the energy, produces a focused energy beam whose intensity increases as its cross sectional area decreases towards a focal point in the target area. At the focal point the area of the focused energy beam is smallest, and intensity is maximal.

An analogy to the concept of how TFED works may be drawn between the TFED destroying the tissue in the target area and sunrays passing through a magnifying glass and burning a spot on a piece of paper. When the magnifying glass is held at a correct angle with respect to the sun, the sunrays go through the lens and intersect at a focal point, which, if the point is on the paper, will cause the paper to burn at the point. If one inserts their hand in the region between the lens and the focal point, no significant heat will be felt nor will harm be caused. Nevertheless, if the hand is placed at the focal point, the hand will be burnt. In TFED, the transducer acts like the magnifying glass and energy, such as, for example ultrasound, is used instead of light. The energy is generally not felt by the tissue in the region between the focal point and the transducer, but is strongly felt by the tissue at the focal point (in the target area).

Although the intensity of the focused energy is maximal at the focal point, in reality the intensity is not zero in regions outside the target area (non-target regions). For example, such may be the case in the tissue between the transducer and the target area. Another example may be in tissue outside the target area and lying in the same plane as the target area (focal plane), and/or even in tissue lying beyond the target area. Occasionally, the intensity of the focused energy in some spots in the non-target regions is sufficiently high so as to cause damage and/or pain to tissue at the spot locations if an accumulated time of exposure to the relatively high intensity is sufficiently long. These spots are known in the art as “secondary hot spots”. For example, tissue in secondary hotspots may not suffer damage after a period of time T, but damage may occur after a period of time of 2T. Secondary hotspots may have adverse side-effects in a therapeutic treatment, possibly producing pain in a patient and, occasionally, burns. Consequently, secondary hot spots place a constraint on the overall intensity which may be delivered to a tissue, and on the intensity which may be reached in the target area where the therapeutic treatment is desired.

Techniques and apparatus are known in the art, which attempt to solve the problem of the secondary hot spots. Some are identified below:

U.S. Pat. No. 4,865,042 “ULTRASONIC IRRADIATION SYSTEM”, discloses “a system for irradiating sound waves to be converged into an annular focal zone having a desired size. This system uses a transducer which is composed of a plurality of elements divided at least in a circumferential direction of the face of the transducer so that the phases of drive signals may be changed according to the respective circumferential positions of the oscillating elements to rotate the phases of the drive signals in rotations in the circumferential direction. As a result, the annular focal zone of having a desired radius is formed, and integrated values of sound waves in the circumferential direction may be substantially zero on the focal plane so that an unnecessary secondary focal zone is prevented from being formed.”

U.S. Pat. No. 5,743,863 “HIGH-INTENSITY ULTRASOUND THERAPY METHOD AND APPARATUS WITH CONTROLLED CAVITATION EFFECT AND REDUCED SIDE LOBES”, discloses “a high-energy ultrasound therapy method and apparatus, said apparatus comprises a therapy device with at least one ultrasound therapy transducer element and a signal generator supplying an electronic signal to said ultrasound transducer element, the signal generator supplying the transducer(s) with a wideband electronic signal of the random or pseudo-random type.”

US Patent Application Publication No. US 2007/0232912 A1 “NON-INVASIVE POSITIONING SYSTEM FOR LOCATING THE FOCUS OF HIGH-INTENSITY FOCUSED ULTRASOUND”, discloses “a non-invasive positioning system for determining the focus location of a HIFU device comprises a diagnostic ultrasound and the HIFU for ablating and removing tumor tissue. The imaging plane of the diagnostic ultrasound probe and the geometrical axis of a probe of the HIFU define an inclining angle during operation. When the imaging plane of the diagnostic ultrasound intersected to the focus of the HIFU transducer, a maximal convergent interference signal was obtained, so as to position the HIFU focus within tumors for precise ablation.”

European Patent Specification EP1274348B1 “SYSTEMS FOR REDUCING SECONDARY HOT SPOTS IN A PHASED ARRAY FOCUSED ULTRASOUND SYSTEM”, discloses “a system for performing a therapeutic procedure in a target tissue region of a patient using focused ultrasound within a primary focal zone, comprising an array of transducer elements; drive circuitry coupled to the transducer elements, the drive circuitry configured to provide respective drive signals to the transducer elements at, at least, first and second discrete frequencies; and a controller coupled to the drive circuitry, characterized in that the controller is configured for periodically changing the frequency of the respective drive signals provided by the drive circuitry between at least the first and second frequencies as often as every 0.2-0.5 seconds while substantially maintaining focus at the primary focal zone during a single sonication.”

### SUMMARY

An aspect of some embodiments of the invention relates to providing an apparatus adapted to transmit focused ultrasound to a treatment area for lysing of adipose tissue. The apparatus may further be adapted to substantially reduce a time averaged intensity of the ultrasound outside the treatment area.

The apparatus may further include a tracking system adapted to track transducer position, and a transducer motion controller adapted to continuously or, optionally, intermittently, according to a predetermined time criteria (for example every 0.1-100 seconds, such as 1-3 seconds, 2-5, seconds or any other time), shift (displace) the position of the transducer. The transducer motion controller is adapted to shift the transducer under the constraint of substantially maintaining the focal point of the focused energy substantially within the target area. In some possible embodiments, the transducer motion controller is further adapted to shift the transducer under another constraint required to maintain appropriate energy transfer into the body. For example, the transducer motion controller may be adapted to ensure physical contact between the transducer and the skin surface of the patient, in addition to preserving the focal point of the focused energy substantially within the target area. Optionally, the transducer may be placed above and at a distance from the skin surface. Optionally, the transducer may be placed inside a container adapted to transfer acoustic energy from the transducer to the skin surface and into a body. Shifting the position of the transducer may comprise lateral translation in 3 dimensions of space, tilting in any direction in space, and/or rotation. The continuous or, optionally, intermittent (according to the predetermined criteria) motion of the transducer, while substantially maintaining transducer focal point within the treatment area, shifts the orientation of the focused energy beam relative to the focal point, and consequently the position of the secondary hotspot in the tissue in the non-target regions. As a result, the time averaged intensity (in other words, the total intensity averaged over a period of time, such as 1-10 seconds, 10-60 minutes) of the focused ultrasound on the secondary hotspot is substantially reduced, preventing damage to tissue in the non-target regions, and enabling the use of increased levels of focused energy in the target area. In some embodiments of the invention, the transducer motion controller is further adapted to move the transducer from a first target area to a second target area.

In an embodiment of the invention, the apparatus comprises a positioning subsystem adapted to allow shifting of the transducer position with 1, 2, 3, 4, 5, or 6 degrees of freedom of movement (up and down, left and right, forward and backward, tilting up and down and/or back and forth, turning left and right, and/or tilting side to side, or any combination thereof) while substantially maintaining the transducer focal point substantially within the target area.

According to some embodiments, the positioning subsystem includes a positioning element. The term positioning element may refer, for example, to one or more elements that facilitate the positioning of the transducer over a subject's body. Optionally, the positioning element includes a mechanical arm, attached to the transducer. The mechanical arm may be adapted to be manually moved by a user (such as a technician), for example, responsive to indications received from the tracking system by visual means and/or audible means.

Optionally, the positioning element includes a robotic arm attached to the transducer. In this case, shifting of the position of the transducer is automatically performed by a transducer motion controller, which may also be considered as a part of the positioning subsystem. The positioning element (such as the robotic arm), which is attached to the transducer, may be automatically moved, responsive to tracking signals received by the transducer motion controller from the tracking system.

In an embodiment of the invention, the transducer may comprise a semi-spherical (concave) shape. Optionally, the transducer may comprise other shapes, for example, a semi-cylindrical shape, flat shape, or any other shape.

In some embodiments of the invention, the focused energy may be static (time constant). In some embodiments of the invention, the focused energy may be time dependent such as, for example, in an arrayed transducer, which comprises one or more transducer elements in the transducer.

There is provided, in accordance with an embodiment of the invention, an apparatus for delivering focused therapeutic energy, the apparatus comprising a transducer adapted to transmit focused energy to a target area tissue of a subject body; and a positioning element adapted to shift the transducer while transmitting the focused energy to the target area tissue, wherein the positioning element is further adapted to substantially maintain a focal point of the transducer within the target area tissue of the subject body. Optionally, the apparatus is adapted to lyse adipose tissue. Optionally, shifting of the transducer is adapted to reduce the time averaged intensity of the focused energy delivered outside the target area tissue of the subject body. Optionally, the focused energy comprises a dynamic focused energy.

In some embodiments of the invention, the focused therapeutic energy comprises ultrasonic energy. Optionally, the transducer is further adapted to maintain acoustic contact between said transducer and a skin surface of the subject body, while the transducer is shifted.

In some embodiments of the invention, the apparatus further comprises a controller adapted to control the shift of the transducer.

Optionally, shifting the transducer comprises 1, 2, 3, 4, 5, or 6 degrees of freedom of movement. Optionally, the apparatus further comprises a tracking system adapted to track the position of said transducer.

In some embodiments of the invention, the focused energy comprises high intensity focused ultrasound (HIFU). Optionally, the focused energy comprises medium intensity focused ultrasound (MIFU). Optionally, the focused energy comprises low intensity focused ultrasound (LIFU).

In some embodiments of the invention, the positioning element comprises a robotic arm. Optionally, the positioning element comprises a mechanical arm. Optionally, the positioning element provides shifting of the transducer with at least one degree of freedom of movement. Optionally, the positioning element is adapted to shift the transducer continuously at a constant rate. Optionally, the positioning element is adapted to shift the transducer continuously at a variable rate. Additionally or alternatively, the positioning element is adapted to shift the transducer intermittently. Optionally, the positioning element is adapted to shift the transducer randomly. Optionally, the positioning element is further adapted to move the transducer from a first target area to a second target area.

In some embodiments of the invention, the transducer comprises a phased array transducer. Optionally, the apparatus further comprises a controller adapted to control the position of the transducer and the focal point relative to the transducer.

In some embodiments of the invention, the apparatus is further adapted to provide visual and/or audible positioning indication.

There is provided, in accordance with an embodiment of the invention, a method for delivering focused therapeutic energy, the method comprising transmitting focused energy from a transducer to a target area tissue of a subject body; shifting the transducer while substantially maintaining a focal point of the transducer within the target area. Optionally, the focused therapeutic energy is adapted to lyse adipose tissue. Optionally, shifting the transducer is adapted to reduce the time averaged intensity of the focused energy delivered outside the target area tissue of the subject body. Optionally, the focused therapeutic energy comprises ultrasonic energy.

In some embodiments of the invention, the method further comprises tracking the position of the transducer.

In some embodiments of the invention, the method further comprises high intensity focused ultrasound (HIFU) as the focused energy. Optionally, the focused energy comprises medium intensity focused ultrasound (MIFU). Optionally, the focused energy comprises low intensity focused ultrasound (LIFU).

In some embodiments of the invention, the method further comprises shifting the transducer with 1, 2, 3, 4, 5, or 6 degrees of freedom of movement. Optionally, the method further comprises automatically shifting the transducer. Additionally or alternatively, shifting the transducer comprises using a robotic arm. Optionally, shifting the transducer includes at least one degree of freedom of movement. Optionally, the method further comprises continuously shifting the transducer at a constant rate. Optionally, the method further comprises continuously shifting the transducer at a variable rate. Additionally or alternatively, the method further comprises intermittently shifting the transducer. Optionally, the method further comprises randomly shifting the transducer.

In some embodiments of the invention, the method comprises controlling simultaneously the location of a focal point relative to the transducer and the position of the transducer relative to subject's body. In this case the transducer is adapted to enable shifting the focal point relative to the transducer, such as in a phased-array transducer.

In some embodiments of the invention, the method further comprises providing positioning visual and/or audible indications.

There is provided, in accordance with an embodiment of the invention, a system for delivering focused therapeutic energy, the system comprising a transducer adapted to transmit focused energy to a target area tissue of a subject body; a positioning element adapted to shift said transducer while transmitting the focused energy to the target area tissue, wherein said positioning element is further adapted to substantially maintain a focal point of said transducer within the target area tissue of the subject body; and a motion controller adapted to control the shift of said transducer. Optionally, the focused therapeutic energy comprises ultrasonic energy. Additionally or alternatively, the system is further adapted to lyse adipose tissue. Optionally, shifting the transducer is adapted to reduce the time averaged intensity of the focused energy delivered outside the target area tissue of the subject body.

In some embodiments of the invention, the focused therapeutic energy comprises ultrasonic energy. Optionally, the transducer is further adapted to maintain acoustic contact between said transducer and a skin surface of the subject body, while the transducer is shifted.

In some embodiments of the invention, the apparatus further comprises a controller adapted to control the shift of the transducer.

Optionally, shifting the transducer comprises 1, 2, 3, 4, 5, or 6 degrees of freedom of movement.

In some embodiments of the invention, the system further comprises a tracking system adapted to track the position of the transducer.

In some embodiments of the invention, the focused energy comprises high intensity focused ultrasound (HIFU). Optionally, the focused energy comprises medium intensity focused ultrasound (MIFU). Optionally, the focused energy comprises low intensity focused ultrasound (LIFU).

In some embodiments of the invention, the positioning element comprises a robotic arm. Optionally, the positioning element comprises a mechanical arm. Optionally, the positioning element provides shifting of the transducer with at least one degree of freedom of movement. Optionally, the positioning element is adapted to shift the transducer continuously at a constant rate. Optionally, the positioning element is adapted to shift the transducer continuously at a variable rate. Additionally or alternatively, the positioning element is adapted to shift the transducer intermittently. Optionally, the positioning element is adapted to shift the transducer randomly. Optionally, the positioning element is further adapted to move the transducer from a first target area to a second target area.

In some embodiments of the invention, the transducer comprises a phased array transducer. Optionally, the system further comprises a controller adapted to control the position of the transducer and the focal point relative to the transducer.

In some embodiments of the invention, the apparatus is further adapted to provide visual and/or audible positioning indication.

### BRIEF DESCRIPTION OF FIGURES

Examples illustrative of embodiments of the invention are described below with reference to figures attached hereto. In the figures, identical structures, elements or parts that appear in more than one figure are generally labeled with a same numeral in all the figures in which they appear. Dimensions of components and features shown in the figures are generally chosen for convenience and clarity of presentation and are not necessarily shown to scale. The figures are listed below.

FIG. 1 schematically shows an exemplary apparatus comprising an automatic transducer positioning element, in accordance with an embodiment of the invention;

FIG. 2 schematically shows an exemplary apparatus comprising a manual transducer positioning element, in accordance with another embodiment of the invention;

FIG. 3 schematically shows a side view of an exemplary transducer in three positions, in accordance with an embodiment of the invention;

FIG. 3A schematically shows an exemplary secondary hot spot in a patient's tissue, in accordance with an embodiment of the invention;

FIG. 3B schematically shows a displacement of the secondary hot spot of FIG. 3A following shifting of a transducer, in accordance with an embodiment of the invention;

FIG. 3C schematically shows another displacement of the secondary hot spot of FIG. 3A following additional shifting in a transducer in accordance with an embodiment of the invention;

FIG. 4 schematically shows an isometric view of an exemplary transducer comprised in an apparatus, in exemplary shift positions on a skin surface of a patient, in accordance with an embodiment of the invention;

FIG. 4A schematically shows an exemplary secondary hot spot in a patient's tissue, in accordance with another embodiment of the invention;

FIG. 4B schematically shows a displacement of the secondary hot spot of FIG. 4A following shifting of a transducer, in accordance with another embodiment of the invention;

FIG. 4C schematically shows another displacement of the secondary hot spot of FIG. 4A following additional shifting in a transducer, in accordance with another embodiment of the invention;

FIG. 5 schematically shows a side view of an exemplary phased-array transducer in three positions, in accordance with an embodiment of the invention;

FIG. 5A schematically shows an exemplary secondary hot spot in a patient's tissue from the arrayed transducer, in accordance with another embodiment of the invention;

FIG. 5B schematically shows a displacement of the secondary hot spot of FIG. 5A following shifting of the arrayed transducer, in accordance with another embodiment of the invention;

FIG. 5C schematically shows another displacement of the secondary hot spot of FIG. 5A following additional shifting in the arrayed transducer, in accordance with another embodiment of the invention;

FIG. 6 schematically shows a side view of an exemplary phased-array transducer comprised in an apparatus, in exemplary shift positions on a skin surface of a patient, in accordance with an embodiment of the invention;

FIG. 6A schematically shows an exemplary first secondary hot spot in a patient's tissue from an arrayed transducer, in accordance with another embodiment of the invention;

FIG. 6B schematically shows a different secondary hot spot from the arrayed transducer in FIG. 6A, in accordance with another embodiment of the invention; and

FIG. 6C schematically shows another secondary hot spot from the arrayed transducer in FIG. 6A, in accordance with another embodiment of the invention.

### DETAILED DESCRIPTION

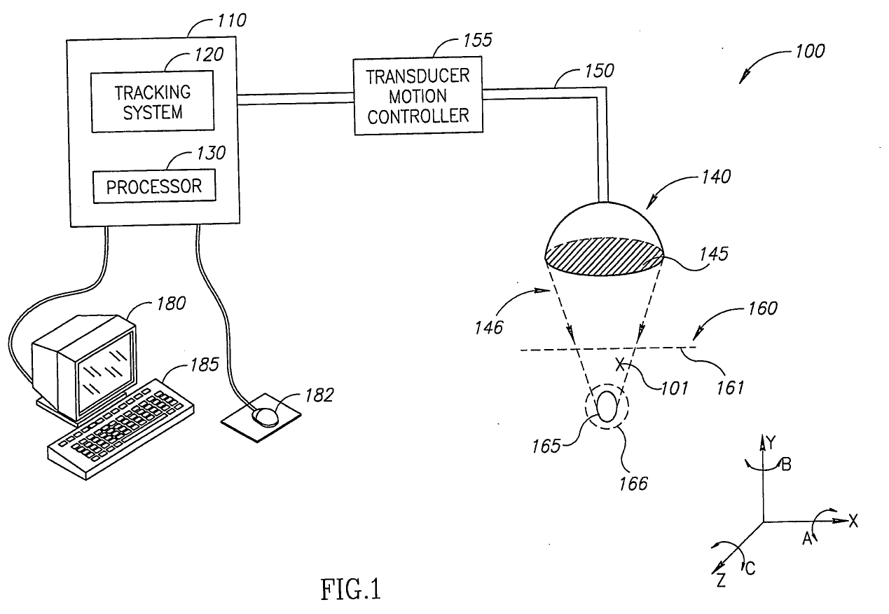

Reference is made to FIG. 1, which schematically shows an exemplary apparatus for delivering therapeutic focused energy 100, in accordance with an embodiment of the invention. Apparatus 100 comprises a base unit 110, which includes a tracking system 120 and a processor 130; a transducer 140 which connects to base unit 110 through a positioning element such as a robot arm 150; a transducer motion controller 155; an optional display 180; and optional data input means such as a keyboard 185 and a mouse 182.

Transducer 140 is adapted to radiate focused energy to a body 161 of a patient 160. The primary hot spot of a transducer element 145 is located in its focal point 165. Focal point 165 location relative to transducer element 145 may be controllable and may shift with time. In an embodiment of the invention, transducer 140 may be adapted to generate ultrasound energy. Transducer 140 may comprise one or more transducer elements 145, the transducer adapted to convert electrical signals (electrical energy) from processing unit 130 into ultrasound. Focusing of the ultrasound may be achieved by a geometrical design of transducer element 145, and/or by electrical characteristics of the electrical signals.

Processor 130 is adapted to send electrical energy to transducer 140. Additionally, processor 130 is adapted to cool transducer 140, and is further adapted to control transducer 140 so as to ensure effective focused energy transmission to target area 166, including triggering of transducer 140.

Tracking system 120 is adapted to generate feedback about a position of focal point 165 relative to target area 166. The feedback is processed by motion controller 155 to appropriately move transducer 140 such that focal point 165 will be correctly positioned relative to target area 166. Position of focal point 165 may be determined by tracking the position of transducer 140 together with stored information about the location of the focal point relative to the transducer. In some embodiments, the location of focal point 165 relative to transducer 140 may vary with time. The position of target area 166 may be achieved by MRI, x-ray, imaging ultrasound, or other imaging modality. Also, it may be determined by acquiring an image of the body, combined with stored information about the location of the treatment area relative to the body.

In accordance with an embodiment of the invention, tracking system 120 is further adapted to send tracking signals to transducer motion controller 155. The tracking signals comprise control signals associated with shifting the position of transducer 140 with respect to target area 166, while substantially maintaining focal point 165 within target area 166. In accordance with an embodiment of the invention, shifting the position of transducer 140 shifts an orientation of transducer element 145, and thereby shifts the position of secondary hot spots 101 in non-target regions. Shifting the position of unit 140 may be performed continuously, at a constant speed or, optionally, at a variable speed. Optionally, shifting transducer 140 may be performed intermittently, according to a predetermined time criteria. Shifting the position of transducer 140 may comprise transducer displacement with 1, 2, 3, 4, 5, or 6 degrees of freedom. Displacement may be along an x-axis, y-axis, and/or z-axis; tilting by partial or full rotation around the x-axis as shown by A (roll), y-axis as shown by B (yaw), and/or z-axis as shown by C (pitch); or any combination thereof. Optionally, the tracking signals may additionally comprise control signals associated with correctly positioning transducer 140 with respect to target area 166, as may be required for proper focusing of focal point 165 within target area 166. Optionally, the tracking signals may additionally comprise control signals associated with moving transducer 140 from one target area to a next target area.

In accordance with an embodiment of the invention, transducer motion controller 155, responsive to tracking signals received from tracking system 120, is adapted to shift robot arm 150 finite distances along the x, y, and/or z axes, and optionally roll, yaw and/or pitch, or any combination thereof. Optionally, transducer motion controller 155 may be further adapted to allow transducer 140 to translate on robot arm 150 with 1, 2, 3, 4, 5, or 6 degrees of freedom. Optionally, transducer motion controller 155 is adapted to move robot arm 150 from one target area to a second target area, and to correctly position transducer 140 over the target area. Transducer motion controller 155 may comprise electric motors, such as, for example, stepper motors, to effect the movement in robot arm 150, and optionally transducer 140. Optionally, in some embodiments of the invention, hydraulic, pneumatic, and/or magnetic means, or any combination thereof, may be used to effect movement in robot arm 150, and optionally transducer 140. Optionally, electric motors may be used in combination with the hydraulic, pneumatic, and/or magnetic means, or any combination thereof.

In accordance with an embodiment of the invention, robot arm 150 is responsive to control signals from motion controller 155. Robot arm 150 is adapted to shift transducer 140 finite distances along the x, y, and/or z axes, and optionally roll, yaw and/or pitch, or any combination thereof. Optionally robot arm may be further adapted to allow transducer 140 to translate on the robot arm with 1, 2, 3, 4, 5, or 6 degrees of freedom. Robot arm 150 may further be adapted to move transducer 140 from one target area to a second target area, and to correctly position transducer 140 over the target area.

Optional display 180, keyboard 185 and mouse 182 are adapted to allow technician input/output data interface with apparatus 100. Information may be displayed in display 180 such as, for example, position of transducer 140 relative to treatment area 166, transducer shift, data on intensity at focal point 165, position of transducer 140 relative to a next treatment area, close up images of treatment area 166, previously stored data, among numerous others. Input data through keyboard 185 and/or mouse 182 may also be displayed in display 180, such as, for example, a predetermined time criteria for intermittent shifts, rate of shift (constant speed or variable), type of shifting (x, y and/or z-axis translation, and/or roll, yaw, pitch, or any combination thereof), among numerous others.

In accordance with an embodiment of the invention, apparatus 100 is adapted to continuously or, optionally intermittently (according to the predetermined time criteria), automatically shift the position of transducer 140 with respect to target area 166 in patient 160, while retaining focal point 165 within target area 166. Continuously, or optionally intermittent, shifting of transducer 140 shifts the orientation of focused energy beam 146 relative to focal point 165, and the position of secondary hotspot 101 in tissue in the non-target region of patient 160. As a result, a time averaged intensity of focused energy beam 146 on secondary hotspot 101 is substantially reduced. Consequently, damage to tissue in the non-target region is substantially prevented, and increased levels of intensity may be used for the target area. Optionally, apparatus 100 may be further adapted to automatically move transducer 140 from one target area to a second target area and to correctly position and shift transducer 140 over the second target area.

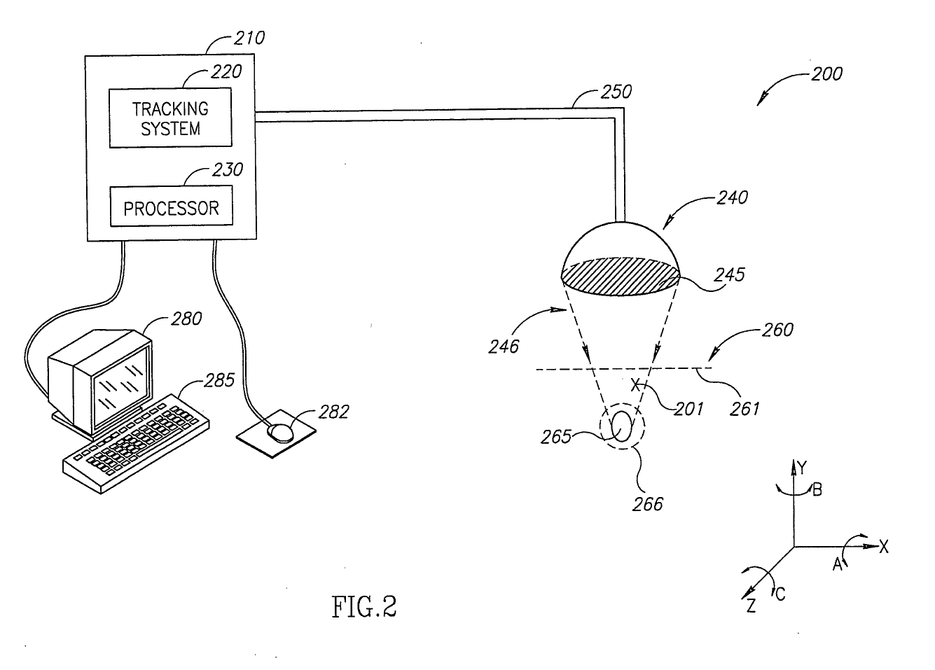

Reference is made to FIG. 2, which schematically shows an exemplary apparatus 200 comprising a manual transducer positioning element 250, in accordance with another embodiment of the invention. Apparatus 200 comprises a base unit 210 which includes a tracking system 220 and a processor 230; a transducer 240, which includes a transducer element 245, and which connects to base unit 210 through a positioning element such as a mechanical arm 250; an optional display 280; and optional data input means such as a keyboard 285 and a mouse 282. Base unit 210, processor 230, transducer 240 including transducer element 245, display 280, keyboard 285 and mouse 282, are the same or substantially similar to that shown in FIG. 1 at 110, 130, 140, 145, 180, 185, and 182.

Tracking system 220 is adapted to generate feedback about a position of-focal point 265 relative to target area 266. Position of focal point 265 may be determined by tracking the position of transducer 240, together with stored information about the location of the focal point relative to the transducer. In some embodiments, the location of focal point 265 relative to transducer 240 may vary with time. The position of target area 266 may be achieved by MRI, x-ray, imaging ultrasound, or other imaging modality. Also, it may be determined by acquiring an image of the body, combined with stored information about the location of the treatment area relative to the body.

In accordance with an embodiment of the invention, tracking system 220 is further adapted to provide visual indications, and optionally aural indications, to a technician. Responsive to the visual and optionally aural indication, the technician is responsible for manually shifting the position of transducer 240 with respect to target area 266, while substantially maintaining focal point 265 within treatment area 266. In accordance with an embodiment of the invention, manual shifting of transducer 240 shifts an orientation of transducer element 245, and thereby shifts the position of secondary hot spots 201 in non-target regions. Shifting the position of transducer 240 may be performed manually by the technician, either through continuous movements or intermittent movements of mechanical arm 250 and/or transducer 240. A constrained motion of transducer 240 comprises substantially maintaining focal point 265 of focused energy beam 246 substantially within target area 266. Shifting the position of transducer 240 may comprise transducer displacement with 1, 2, 3, 4, 5, or 6 degrees of freedom. Displacement may be along an x-axis, y-axis, and/or z-axis; tilting by partial or full rotation around the x-axis as shown by A (roll), y-axis as shown by B (yaw), and/or z-axis as shown by C (pitch); or any combination thereof. Optionally, tracking system 220 may be further adapted to provide visual indications, and optionally aural indications, to the technician related to correctly positioning transducer 240 with respect to target area 266, as may be required for proper focusing of focal point 265 within target area 266. Optionally, tracking system 220 may be further adapted to provide visual indications, and optionally aural indications, to the technician related to moving transducer 240 from one target area to a next target area. Tracking system 220 may be further adapted to trigger warning signals, which may be visually displayed in display 280, or may optionally activate an aural warning device, if possible hazardous conditions arise. Hazardous conditions may be, for example, if shifting of transducer 240 is performed incorrectly or in an untimely manner by the technician. Optionally, for example, if transducer 240 is incorrectly positioned with respect to target area 266, and/or if focal point 265 is outside of the target area. Additionally or alternatively, if transducer 240 is moved from one target area to an incorrect second target area.

In accordance with an embodiment of the invention, mechanical arm 250 is adapted to allow a technician to manually shift transducer 240 finite distances along the x, y, and/or z axes, and optionally roll, yaw and/or pitch, or any combination thereof. Optionally, mechanical arm 250 may be further adapted to allow transducer 240 to manually translate on the mechanical arm with 1, 2, 3, 4, 5, or 6 degrees of freedom. Mechanical arm 250 may further be adapted to allow manual movement of transducer 240 from one target area to a second target area, and to manually position the transducer over the target area.

In accordance with an embodiment of the invention, apparatus 200 is adapted to enable a technician to continuously or, optionally intermittently, shift the position of transducer 240 with respect to target area 266 in a body 261 of a patient 260, while substantially maintaining focal point 265 within target area 266. Continuously, or optionally intermittent, shifting of transducer element 245 shifts the orientation of focused energy beam 246 relative to focal point 265, and the position of secondary hotspot 201 in tissue in the non-target region of patient 260. As a result, a time averaged intensity of focused energy beam 246 on secondary hotspot 201 is substantially reduced. Consequently, damage to tissue in the non-target region is substantially prevented, and increased levels of intensity may be used for the target area. Optionally, apparatus 200 may be further adapted to allow a technician to manually move transducer 240 from one target area to a second target area and to correctly position and shift the transducer over the second target area. Apparatus 200 may be further adapted to issue visual and/or aural warnings to the technician when hazardous conditions arise.

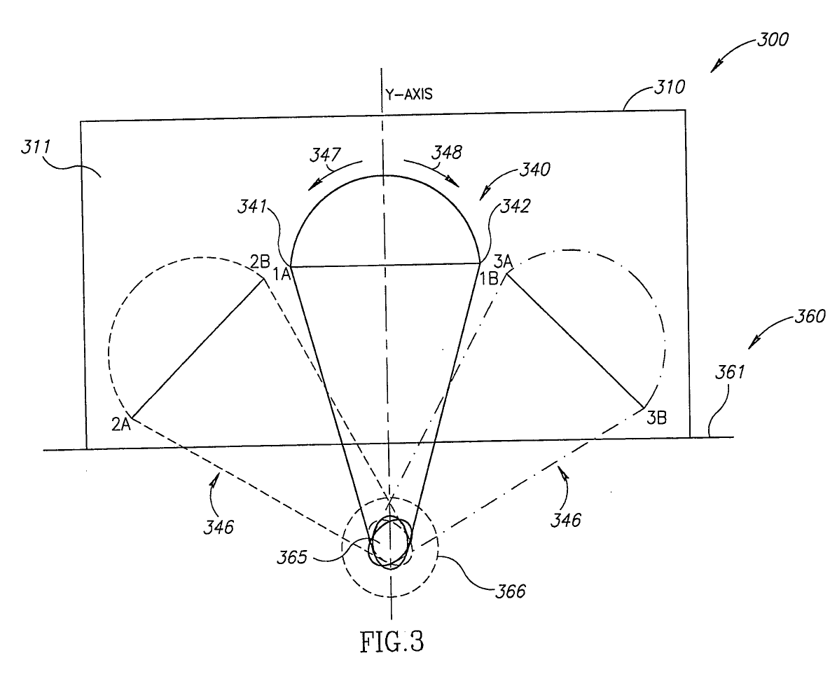

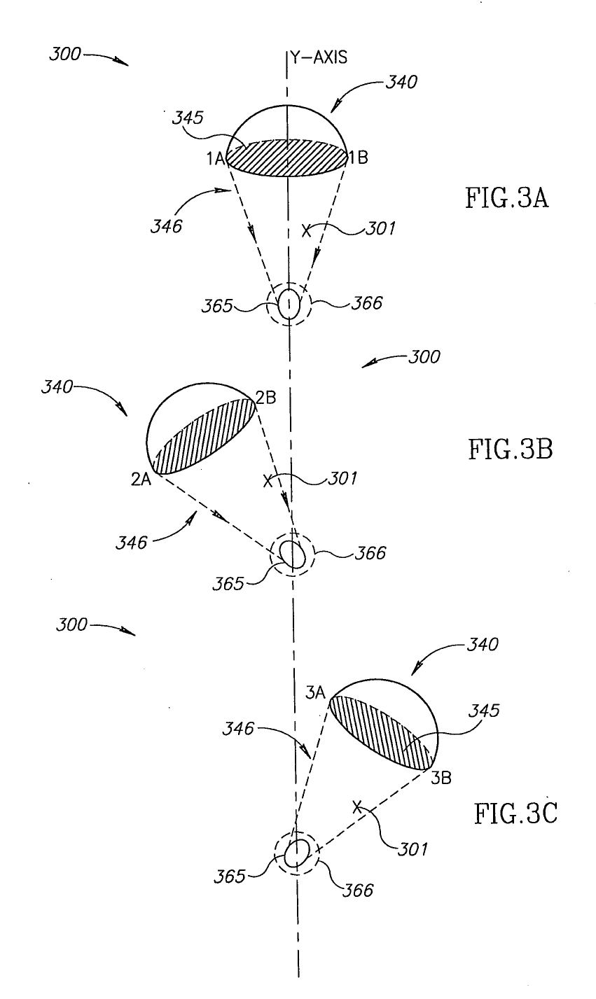

Reference is made to FIG. 3, which schematically shows a side view of an exemplary transducer 340 comprised in an apparatus 300, in exemplary shift positions on the body 361 of a patient 360, in accordance with an embodiment of the invention. Apparatus 300 may be the same or substantially similar to that shown in FIG. 1 at 100, including transducer 340, which may be the same or substantially similar to that shown in FIG. 1 at 140. Optionally, apparatus 300 may be the same or substantially similar to that shown in FIG. 2 at 200, including transducer 340 which may be the same or substantially similar to that shown in FIG. 2 at 240.

Transducer 340 is shown in a first position wherein the transducer is placed inside a container 310 comprising an acoustic coupling medium 311, such as for example oil, the container flatly placed against the body 361. Transducer edges 341 and 342 are shown in positions 1A and 1B inside acoustic coupling medium 311, respectively. Focused energy beam 346 is substantially concentrated at a focal point 365, in a target area 366. Transducer 340 is shifted in position by a rolling or pitching motion about a y-axis, in a direction shown by curved arrow 347, such that edges 341 and 342 move into new positions 2A and 2B. This causes a shift in a direction of focused energy beam 346 while maintaining focal point 365 within target area 366. Transducer 340 is shifted in position by a rolling or pitching motion about a y-axis, in a direction shown by curved arrow 348, such that edges 341 and 342 move into new 3A and 3B. This causes a shift in a direction of focused energy beam 346 while maintaining focal point 365 within target area 366. Existence of one or more hot spots and hot spot locations may relate to transducer characteristics, tissue characteristics, a combination of them, or else. Therefore, shifting transducer 340 under the constraint of substantially maintaining focal point 365 within target area 366 may result in shifting hot spots in tissue, changing the intensity of the hot spots or even appearance or disappearance of hot spots.

Reference is also made to FIGS. 3A, 3B, and 3C which schematically show detailed views of the shifting of position of an exemplary secondary hot spot 301 as transducer 340 in FIG. 3 is shifted in position, in accordance with an embodiment of the invention.

In FIG. 3A, transducer 340 is placed over target area 366 and focused energy beam 346 is transmitted from transducer element 345, such that focal point 365 is located in the target area. Secondary hot spot 301 appears in patient's 360 tissue as a result of relatively high energy in focused energy beam 346 at the location of the secondary hot spot, which is located in a non-target region, in tissue between target area 366 and transducer 340. Optionally, secondary hot spot 301 may be located in another non-target region, and/or there may a plurality of secondary hot spots.

In FIG. 3B, transducer 340 is shifted in the direction of curved arrow 347 resulting in focused energy beam 346 shifting in the same direction as curved arrow 347, and focal point 365 is located in target area 366. In accordance with an embodiment of the invention, the relatively high energy spot in focused energy beam 346 may maintain its position relative to the transducer within the focused energy. Shifting the focused energy beam by shifting transducer 340 in the direction of curved arrow 347 shifts secondary hot spot 301 tissue in the direction of curved arrow 347 to a new location in the patient's tissue. Additionally, transducer-tissue interaction may create a new set of hot spots with small probability to hit the same position within tissue.

In FIG. 3C, transducer 340 is shifted in the direction of curved arrow 348, resulting in focused energy beam 346 shifting in the same direction as curved arrow 348, and focal point 365 is located in target area 366. In accordance with an embodiment of the invention, the relative high energy spot in focused energy beam 346 maintains its position within the focused energy, and shifting the focused energy by shifting transducer 340 in the direction of curved arrow 348 shifts secondary hot spot 301 tissue in the direction of curved arrow 348 to a new location in the patient's tissue.

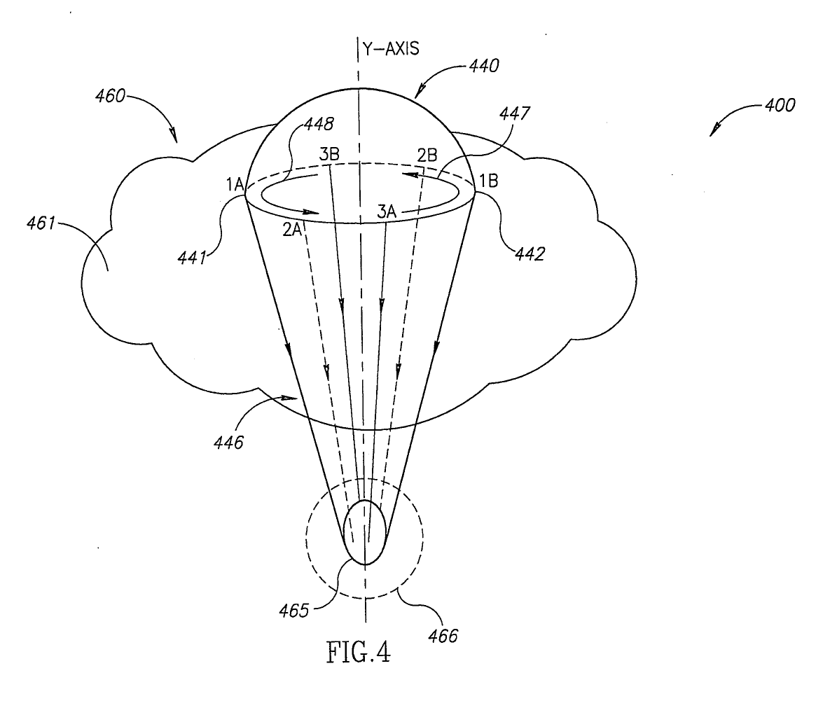

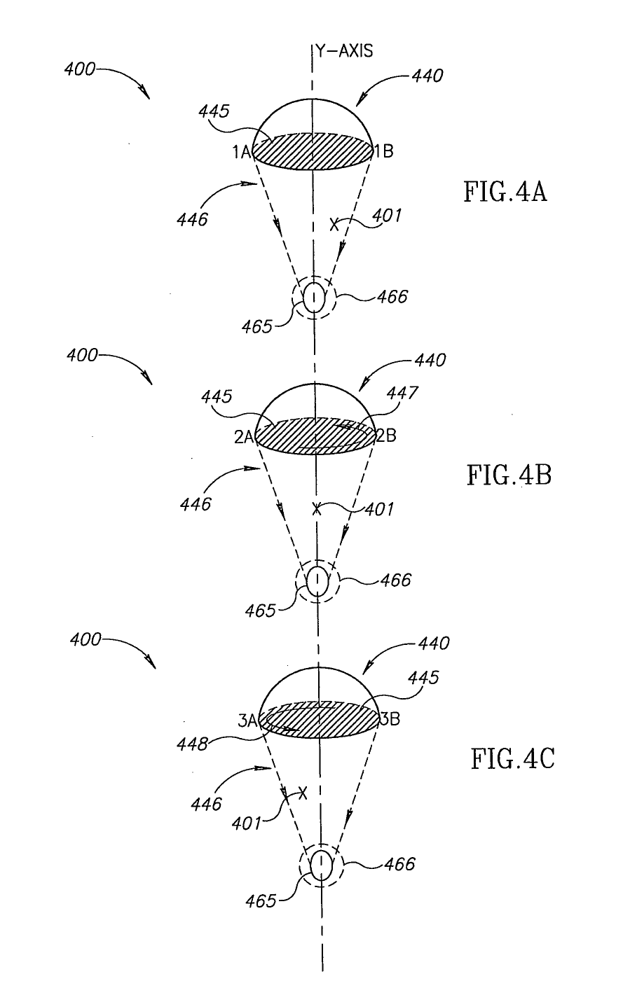

Reference is made to FIG. 4, which schematically shows an isometric view of an exemplary transducer 440 comprised in n apparatus 400, in exemplary shift positions on a skin surface 461 of a patient 460, in accordance with another embodiment of the invention. Apparatus 400 may be the same or substantially similar to that shown in FIG. 1 at 100, including transducer 440, which may be the same or substantially similar to that shown in FIG. 1 at 140. Optionally, apparatus 400 may be the same or substantially similar to that shown in FIG. 2 at 200, including transducer 440, which may be the same or substantially similar to that shown in FIG. 2 at 240.

Transducer 440 is shown in a first position wherein the transducer is flatly placed against skin surface 461, and transducer edges 441 and 442 are shown in positions 1A and 1B on the skin surface, respectively. Focused energy 446 is substantially concentrated at focal point 456 in target area 466. Transducer 440 is shifted in position by a rotational (yaw) motion about a y-axis, in a direction shown by curved arrow 447, such that edge 441 rotates to a new position 2A, and edge 442 rotates to a new position 2B. This causes a rotational displacement of focused energy beam 446 about the y-axis and, as a result, focal point 465 also undergoes a rotational displacement. Transducer 440 is subject to another shift in position by a rotational (yaw) motion about the y-axis, in a direction shown by curved arrow 448, such that edge 441 rotates to a new position 3A, and edge 442 rotates to a new position 3B. This causes an additional rotational displacement of focused energy beam 446 about the y-axis, and also of focal point 465.

Existence of one or more hot spots and hot spot locations may relate to transducer element characteristics, tissue characteristics, combination of them, or else. Therefore, shifting transducer 440 under the constraint of substantially maintaining focal point 465 within target area 466 may result in shifting hot spots in tissue, changing the intensity of the hot spot or even appearance or disappearance of hot spots.

Reference is also made to FIGS. 4A, 4B, and 4C, which schematically show detailed views of the shifting of position of an exemplary secondary hot spot 401 as transducer 440 in FIG. 4 is shifted in position, in accordance with another embodiment of the invention.

In FIG. 4A, transducer 440 is placed over target area 466 and focused energy beam 446 transmitted from transducer element 445 such that focal point 465 is centered on the y-axis in the target area. Secondary hot spot 401 appears in patient's 461 tissue as a result of relatively high energy in focused energy beam 446 at the location of the secondary hot spot, located in a non-target region, in tissue between target area 466 and transducer 440. Optionally, secondary hot spot 401 may be elsewhere in another non-target region, and/or there may a plurality of secondary hot spots.

In FIG. 4B, transducer 440 is shifted in the direction of curved arrow 447 resulting in focused energy beam 446 rotating in the same direction as curved arrow 447, and focal point 465 rotating about the y-axis. In accordance with another embodiment of the invention, the relative high energy spot in focused energy beam 446 maintains its position within the focused energy, and rotating the focused energy by shifting transducer 440 in the direction of curved arrow 447 rotates secondary hot spot 401 tissue in the direction of curved arrow 447 to a new location in the patient's tissue.

In FIG. 4C, transducer 440 is shifted in the direction of curved arrow 448 resulting in focused energy beam 446 rotating in the same direction as curved arrow 448, and focal point 465 rotating about the y-axis. In accordance with another embodiment of the invention, the relative high energy spot in focused energy beam 446 maintains its position within the focused energy, and rotating the focused energy by shifting transducer 440 in the direction of curved arrow 448 rotates secondary hot spot 401 tissue in the direction of curved arrow 448 to a new location in the patient's tissue.

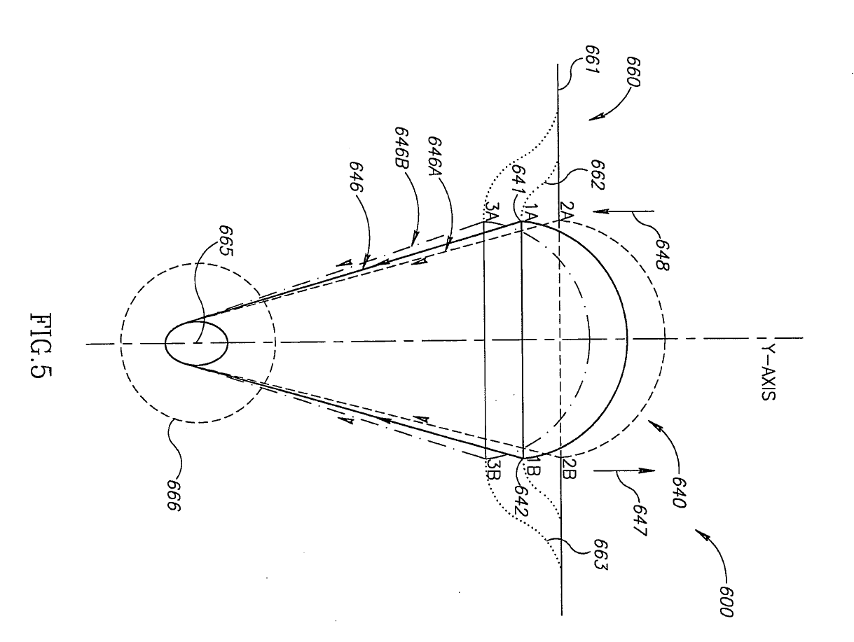

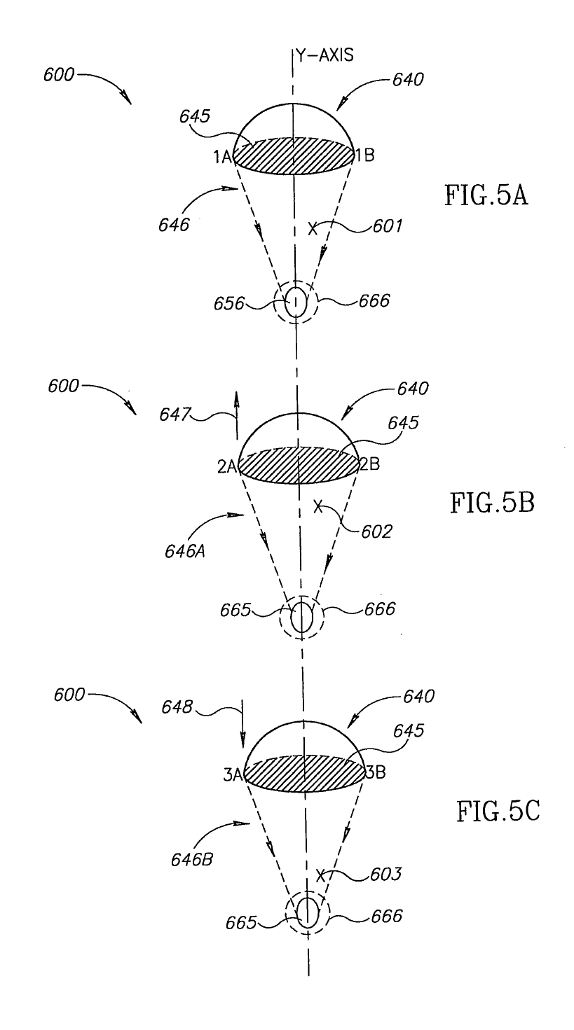

Reference is made to FIG. 5, which schematically shows a side view of an exemplary phased-array transducer 640 comprised in an apparatus 600, in exemplary shift positions on a skin surface 661 of a patient 660, in accordance with another embodiment of the invention. Apparatus 600 may be the same or substantially similar to that shown in FIG. 1 at 100, including transducer 640 which may be the same or substantially similar to that shown in FIG. 1 at 140. Optionally, apparatus 600 may be the same or substantially similar to that shown in FIG. 2 at 200, including transducer 640 which may be the same or substantially similar to that shown in FIG. 2 at 240.

Transducer 640 is shown in a first position wherein the transducer is flatly pressed against skin surface 661, creating an indentation of the skin surface (as shown by hatched lines 662). Transducer edges 641 and 642 are shown in positions 1A and 1B on the indented skin surface, respectively. Focused energy beam 646, the focused energy originating from one or more transducer elements (not shown) comprised in transducer 640, is substantially concentrated at a focal point 665, shown in a target area 666. Transducer 640 is shifted in position by a vertical upward displacement in a direction shown by arrow 647, such that edge 641 is displaced from position 1A to a position 2A while edge 642 is displaced from position 1B to a position 2B. In order to maintain the energy concentrated within target area 666, a new focused energy beam 646A is directed towards the focal point by phased-array transducer 640. Energy from focused energy beam 646A is concentrated in focal point 665. Transducer 640 is shifted in position by a vertical downward displacement in a direction shown by arrow 648 such that edge 641 is displaced from position 1A to a position 3A, while edge 642 is displaced from position 1B to a position 3B. In order to maintain energy concentrated within target area 666 a new focused energy beam 646B is directed towards the focal point by phased-array transducer 640. Focused energy beam 646B is concentrated in focal point 665.

Existence of one or more hot spots and hot spot locations may relate to transducer element characteristics, tissue characteristics, a combination of them, or else. Therefore, shifting transducer 640, under the constraint of substantially maintaining focal point 665 within target area 666, may result in shifting hot spots in tissue, changing the intensity of the hot spots or even appearance or disappearance of hot spots.

Reference is also made to FIGS. 5A, 5B, and 5C, which schematically show detailed views of the shifting of position of an exemplary secondary hot spot 601 as transducer 640 in FIG. 5 is shifted in position, in accordance with another embodiment of the invention.

In FIG. 5A, transducer 640 is placed over target area 666, and focused energy beam 646 transmitted from transducer element 645, such that focal point 665 is located in target area 666. Focused energy beam 646 is transmitted from one or more array elements (not shown) in transducer element 645. Secondary hot spot 601 appears in patient's 660 tissue as a result of relatively high energy in focused energy beam 646 at the location of the secondary hot spot, which is located in a non-target region, in tissue between target area 666 and transducer 640. Optionally, secondary hot spot 601 may be elsewhere in another non-target region, and/or there may a plurality of secondary hot spots.

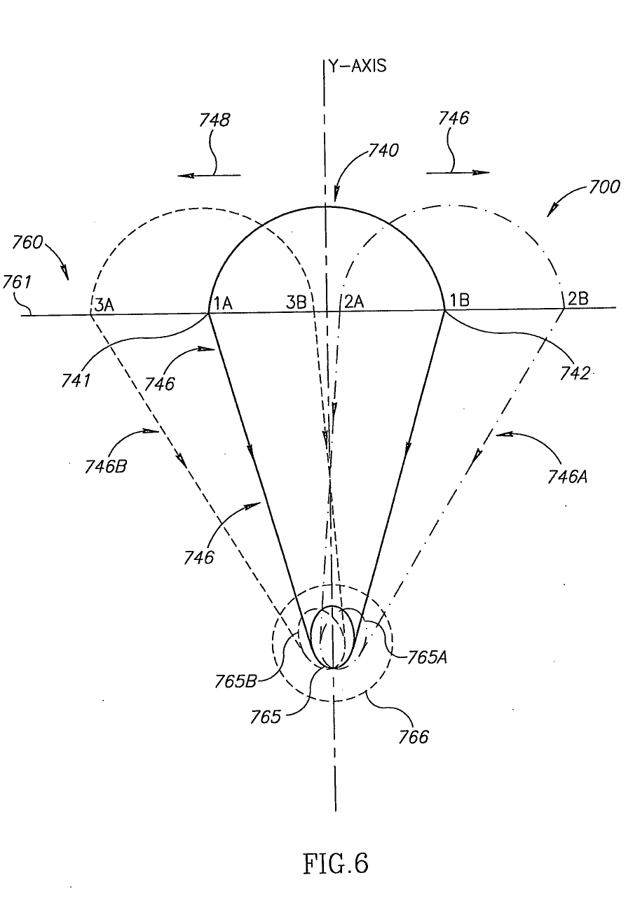

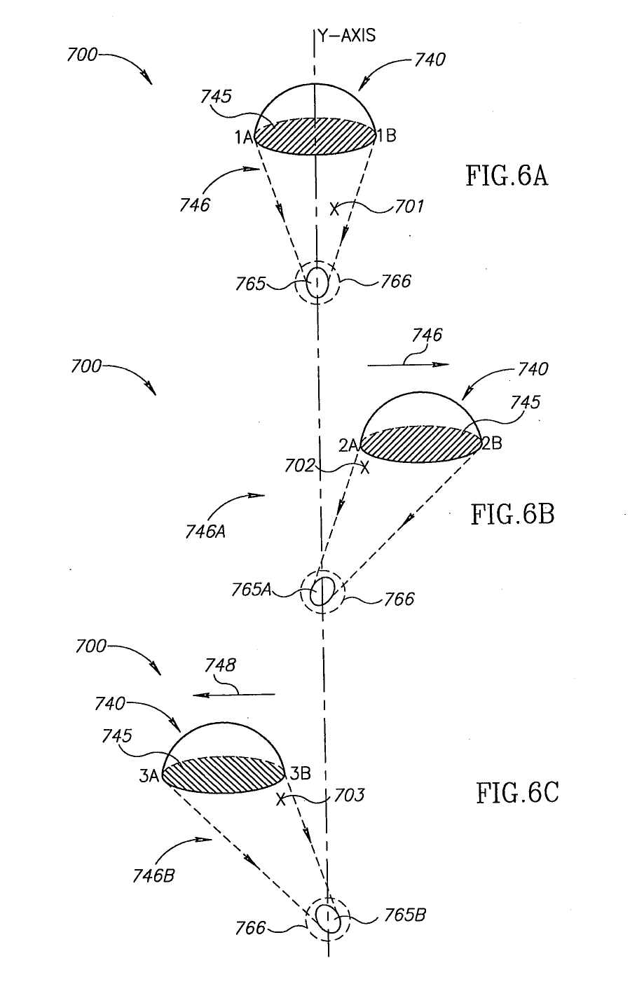

In FIG. 5B, transducer 640 is upwardly shifted in the direction of arrow 647, resulting in focused energy beam 646 vertically shifting in the same direction as arrow 647, and focal point 665 is located in target area 666. Focused energy beam 646A is transmitted from one or more array elements in transducer array 645 different than the array elements used to transmit focused energy beam 646. Optionally, some of the array elements may be the same as those used to transmit focused energy beam 646. In accordance with an embodiment of the invention, focused energy beam 646A is substantially different from focused energy beam 646 and may therefore comprise different relatively high energy spots than those in focused energy beam 646. Consequently, secondary hot spot 601 does not appear in the location shown in FIG. 5A in the patient's tissue, and instead there is a different secondary hot spot 602 in another location. The location of secondary hot spot 602 is shown in another non-target region, in tissue between target area 666 and transducer 640. Optionally, hot spot 601 may be in another non-target region, and/or there may be more than one secondary hot spot. In FIG. 5C, transducer 640 is downwardly shifted in the direction of arrow 648 resulting in focused energy beam 646 vertically shifting in the same direction as arrow 648 such that focal point 665 is located in target area 666. Focused energy beam 646B is transmitted from one or more array elements in transducer array 645 different than the array elements used to transmit focused energy beam 646. Optionally, some of the array elements may be the same as those used to transmit focused energy beam 646. In accordance with an embodiment of the invention, focused energy beam 646B is substantially different to focused energy beam 646 and may therefore comprise different relatively high-energy spots than those in focused energy beam 646. Consequently, secondary hot spot 601 does not appear in the location shown in FIG. 6A in the patient's tissue, and instead there is a different secondary hot spot 603 in another location. The location of secondary hot spot 603 is shown in another non-target region, in tissue between target area 666 and transducer 640. Optionally, hot spot 601 may be elsewhere in another non-target region, and/or there may be more than one secondary hot spot.

Reference is made to FIG. 6, which schematically shows a side view of an exemplary phased-array transducer 740 comprised in apparatus 700, in exemplary shift positions on a skin surface 761 of a patient 760, in accordance with another embodiment of the invention. Apparatus 700 may be the same or substantially similar to that shown in FIG. 1 at 100, including transducer 740, which may be the same or substantially similar to that shown in FIG. 1 at 140. Optionally, apparatus 700 may be the same or substantially similar to that shown in FIG. 2 at 200, including transducer 740, which may be the same or substantially similar to that shown in FIG. 2 at 240.

Transducer 740 is shown in a first position wherein the transducer is flatly placed against skin surface 761 and transducer edges 741 and 742 are shown in positions 1A and 1B on the skin surface, respectively. Focused energy beam 746, the focused energy originating from one or more transducer elements (not shown) comprised in transducer 740, is substantially concentrated at a focal point 765, shown in a target area 766. Transducer 740 is shifted in position by a lateral displacement in a direction shown by arrow 747 such that edge 741 is displaced from position 1A to a position 2A while edge 742 is displaced from position 1B to a position 2B. In order to maintain the energy concentrated within target area 766, a new focused energy beam 746A is directed towards the focal point by phased-array transducer 740. Energy from focused energy beam 746A is concentrated in a new focal point 765A, which may be the same or substantially similar to focal point 765. Optionally, focal point 765A within target area 766 may be different from focal point 765. Transducer 740 is shifted in position by a lateral displacement in a direction shown by arrow 748 such that edge 741 is displaced from position 1A to a position 3A while edge 742 is displaced from position 1B to a position 3B. In order to maintain energy concentrated within target area 766 a new focused energy beam 746B is directed towards the focal point by phased-array transducer 740. Focused energy beam 746B is concentrated in a new focal point 765B, which may be the same or substantially similar to focal point 765. Optionally, focal point 765B within target area 766 may be different from focal point 765.

Existence of one or more hot spots and hot spot locations may relate to transducer element characteristics, tissue characteristics, a combination of them, or else. Therefore, shifting transducer 740, under the constraint of substantially maintaining focal point 765 within target area 766, may result in shifting hot spots 701 in tissue, changing the intensity of the hot spots or even appearance or disappearance of hot spots.

Reference is also made to FIGS. 6A, 6B, and 6C, which schematically show detailed views of different exemplary secondary hot spots 701, 702 and 703, respectively, as phased-array transducer 740 in FIG. 6 is shifted in position, in accordance with another embodiment of the invention.

In FIG. 6A, transducer 740 is correctly placed over target area 766 and focused energy beam 746, transmitted from transducer array 745, such that focal point 765 is located in target area 766. Focused energy beam 746 is transmitted from one or more array elements (not shown) in transducer element 745. Secondary hot spot 701 appears in the patient's tissue as a result of relatively high energy in focused energy beam 746 at the location of the secondary hot spot, which is located in a non-target region, in tissue between target area 766 and transducer 740. Optionally, hot spot 701 may be in another non-target region, and/or there may be more than one secondary hot spot.

In FIG. 6B, transducer 740 is laterally shifted in the direction of arrow 747. Focused energy beam 746A is transmitted such that focal point 765A is located in target area 766. Focused energy beam 746A is transmitted from one or more array elements in transducer array 745 different than the array elements used to transmit focused energy beam 746. Optionally, some of the array elements may be the same as those used to transmit focused energy beam 746. In accordance with an embodiment of the invention, focused energy beam 746A is substantially different from focused energy beam 746 and may therefore comprise different relatively high energy spots than those in focused energy beam 746. Consequently, secondary hot spot 701 does not appear in the location shown in FIG. 7A in the patient's tissue, and instead there is a different secondary hot spot 702 in another location. The location of secondary hot spot 702 is shown in another non-target region, in tissue between target area 766 and transducer 740. Optionally, hot spot 701 may be in another non-target region, and/or there may be more than one secondary hot spot.

In FIG. 6C, transducer 740 is laterally shifted in the direction of arrow 748. Focused energy beam 746B is transmitted such that focal point 765B is located in target area 766. Focused energy beam 746B is transmitted from one or more array elements in transducer array 745, different than the array elements used to transmit focused energy beam 746. Optionally, some of the array elements may be the same as those used to transmit focused energy beam 746. In accordance with an embodiment of the invention, focused energy beam 746B is substantially different from focused energy beam 746 and may therefore comprise different relatively high energy spots than those in focused energy beam 746. Consequently, secondary hot spot 701 does not appear in the location shown in FIG. 6A in the patient's tissue, and instead there is a different secondary hot spot 703 in another location. The location of secondary hot spot 703 is shown in another non-target region, in tissue between target area 766 and transducer 740. Optionally, hot spot 701 may be elsewhere in another non-target region, and/or there may be more than one secondary hot spot.

The location of the secondary hot spots, as shown for example in FIGS. 3-6, is substantially changing with time, while the focus remains substantially within the target area. Thus, the time-averaged intensity outside the target area is reduced.

The examples shown in FIGS. 3A-6A, 3B-6B, and 3C-6C are not intended to be limiting in any form or manner, and are presented for explanatory purposes. A person skilled in the art may appreciate that there are a large number of ways in which the transducer may be shifted, while substantially maintaining acoustic contact. Transducer displacement may comprise 1, 2, 3, 4, 5, or 6 degrees of freedom, and therefore the transducer may be shifted along an x-axis, y-axis, and/or z-axis in an xyz space; tilted by partial or full rotation around the x-axis (roll), y-axis (yaw), and/or z-axis (pitch); or any combination thereof.

In the description and claims of embodiments of the present invention, each of the words, “comprise” “include” and “have”, and forms thereof, are not necessarily limited to members in a list with which the words may be associated.

The invention has been described using various detailed descriptions of embodiments thereof that are provided by way of example and are not intended to limit the scope of the invention. The described embodiments may comprise different features, not all of which are required in all embodiments of the invention. Some embodiments of the invention utilize only some of the features or possible combinations of the features. Variations of embodiments of the invention that are described and embodiments of the invention comprising different combinations of features noted in the described embodiments will occur to persons with skill in the art.

What is claimed is:

1. An apparatus for delivering focused therapeutic energy, the apparatus comprising:
a transducer adapted to transmit focused energy to a target area tissue of a subject body; and
a positioning element adapted to shift said transducer while transmitting the focused energy to the target area tissue, wherein said positioning element is further adapted to substantially maintain a focal point of said transducer within the target area tissue of the subject body.

2. The apparatus according to claim 1, wherein the focused therapeutic energy comprises ultrasonic energy.

3. The apparatus according to claim 2, wherein the transducer is further adapted to maintain acoustic contact between said transducer and a skin surface of the subject body, while the transducer is shifted.

4. The apparatus according to claim 1, adapted to lyse adipose tissue.

5. The apparatus according to claim 1, wherein shifting said transducer is adapted to reduce the time averaged intensity of the focused energy delivered outside the target area tissue of the subject body.

6. The apparatus according to claim 1, further comprising a controller adapted to control the shift of said transducer.

7. The apparatus according to claim 1, further comprising a tracking system adapted to track the position of said transducer.

8. The apparatus according to claim 1, wherein the focused energy comprises high intensity focused ultrasound (HIFU).

9. The apparatus according to claim 1, wherein the focused energy comprises medium intensity focused ultrasound (MIFU).

10. The apparatus according to claim 1, wherein the focused energy comprises low intensity focused ultrasound (LIFU).

11. The apparatus according to claim 1, wherein shifting said transducer comprises 1, 2, 3, 4, 5, or 6 degrees of freedom of movement.

12. The apparatus according to claim 1, wherein the positioning element comprises a robotic arm.

13. The apparatus according to claim 1, wherein said positioning element comprises a mechanical arm.

14. The apparatus according to claim 1, wherein said positioning element provides shifting of the transducer with at least one degree of freedom of movement.

15. The apparatus according to claim 1, wherein said positioning element is adapted to shift the transducer continuously at a constant rate.

16. The apparatus according to claim 1, wherein said positioning element is adapted to shift the transducer continuously at a variable rate.

17. The apparatus according to claim 1, wherein said positioning element is adapted to shift the transducer intermittently.

18. The apparatus according to claim 1, wherein said positioning element is adapted to shift the transducer randomly.

19. The apparatus according to claim 1, wherein said positioning element is further adapted to move the transducer from a first target area to a second target area.

20. The apparatus according to claim 1, wherein said focused energy comprises a dynamic focused energy.

21. The apparatus according to claim 1 wherein said transducer comprises a phased array transducer.

22. The apparatus according to claim 1, further comprising a controller adapted to control the position of said transducer and the focal point relative to transducer.

23. The apparatus according to claim 1, further adapted to provide visual and/or audible positioning indication.

24. A method for delivering focused therapeutic energy, the method comprising:
transmitting focused energy from a transducer to a target area tissue of a subject body; and
shifting the transducer while substantially maintaining a focal point of the transducer within the target area.

25. The method according to claim 24, wherein the focused therapeutic energy comprises ultrasonic energy.

26. The method according to claim 24, wherein the focused therapeutic energy is adapted to lyse adipose tissue.

27. The method according to claim 24, wherein shifting said transducer is adapted to reduce the time averaged intensity of the focused energy delivered outside the target area tissue of the subject body.

28. The method according to claim 24, further comprising tracking the position of the transducer.

29. The method according to claim 24, wherein the focused energy comprises high intensity focused ultrasound (HIFU).

30. The method according to claim 24, wherein the focused energy comprises medium intensity focused ultrasound (MIFU).

31. The method according to claim 24, wherein the focused energy comprises low intensity focused ultrasound (LIFU).

32. The method according to claim 24, wherein shifting said transducer comprises 1, 2, 3, 4, 5, or 6 degrees of freedom of movement.

33. The method according to claim 24, further comprising automatically shifting said transducer.

34. The method according to claim 24, wherein shifting the transducer comprises using a robotic arm.

35. The method according to claim 24 comprising shifting the transducer with at least one degree of freedom of movement.

36. The method according to claim 24 comprising continuously shifting the transducer at a constant rate.

37. The method according to claim 24 comprising continuously shifting the transducer at a variable rate.

38. The method according to claim 24 comprising intermittently shifting the transducer.

39. The method according to claim 24 comprising randomly shifting the transducer.

40. The method according to claim 24 comprising providing positioning visual and/or audible indications.

41. A system for delivering focused therapeutic energy, the system comprising:
a transducer adapted to transmit focused energy to a target area tissue of a subject body;
a positioning element adapted to shift said transducer while transmitting the focused energy to the target area tissue, wherein said positioning element is further adapted to substantially maintain a focal point of said transducer within the target area tissue of the subject body; and
a motion controller adapted to control the shift of said transducer.

42. The system according to claim 41, wherein the focused therapeutic energy comprises ultrasonic energy.

43. The system according to claim 41, adapted to lyse adipose tissue.

44. The system according to claim 41, wherein shifting said transducer is adapted to reduce the time averaged intensity of the focused energy delivered outside the target area tissue of the subject body.

45. The system according to claim 41, further comprising a tracking system adapted to track the position of said transducer.

46. The system according to claim 41, wherein the focused energy comprises high intensity focused ultrasound (HIFU).

47. The system according to claim 41, wherein the focused energy comprises medium intensity focused ultrasound (MIFU).

48. The system according to claim 41 wherein the focused energy comprises low intensity focused ultrasound (LIFU).

49. The system according to claim 41, wherein shifting said transducer comprises 1, 2, 3, 4, 5, or 6 degrees of freedom of movement.
